(19) United States
(12) Patent Application Publication
(10) Pub. No.: US 2004/0102501 A1
(43) Pub. Date: May 27, 2004

(54) Stable, low free formaldehyde, synergistic antimicrobial compositions of aldehyde donors and dehydroacetic acid

(76) Inventors: Patrick Jay Lutz, Nazareth, PA (US); Ahmed M. Tafesh, Allentown, PA (US)

Correspondence Address: DARBY & DARBY P.C., P. O. BOX 5257, NEW YORK, NY, 10150-5257, US

(21) Appl. No.: 10/644,578

(22) Filed: Aug. 20, 2003

Related U.S. Application Data
(60) Provisional application No. 60/405,036, filed on Aug. 20, 2002.

(51) Int. Cl. A61K 31/4164; A61K 31/19; A61K 31/11

(52) U.S. Cl. 514/389; 514/557; 514/693; 514/369; 514/463

(57) ABSTRACT

The present invention provides a stable, low free formaldehyde, synergistic antimicrobial composition, and a method of inhibiting the growth of or reducing microorganisms by applying the composition. Preferably, the constituents are dimethylol dimethylhydantoin, monomethylol dimethylhydantoin, dimethyl hydantoin, and dehydroacetic acid or its salts thereof. The composition has a free formaldehyde content of less than 0.2% and is beneficial for controlling microbiological and fungal contamination in personal care products, household products and industrial products and systems.

This application claims the priority of U.S. Provisional Application No. 60/405,036, filed on Aug. 20, 2002 which is hereby incorporated hereby by reference in its entirety.

### FIELD OF THE INVENTION

The present invention relates to stable, low free formaldehyde, synergistic antimicrobial mixtures of a first component including one or more aldehyde donor, a second component including a stabilizer; and a third component including dehydroacetic acid or salt thereof.

### BACKGROUND OF THE INVENTION

The need for effective and economical preservative compositions is well known. Many products require the addition of a preservative to protect against contamination and growth of microbes. Examples of such products include personal care products such as shampoos, creams, lotions, cosmetics, and soaps; household products such as laundry detergents, hard surface cleaners, fabric softeners, and various industrial products; such as paint, wood, textiles, adhesives, sealants, leather, rope, paper pulp, plastics, fuel, oil, and rubber and metal working fluids. The control of slime-producing bacterial and fungi in pulp and paper mills and in cooling towers is also a matter of substantial commercial importance.

In particular, personal care product compositions provide a nutrient-rich media which benefit from the incorporation of preservatives to control the growth of microorganisms and to prevent spoilage. Generally, the shelf life of these products depends on the resistance to microbial spoilage of components contained therein. It is therefore desirable to formulate a preservative which controls microbial contamination in personal care products, household products, and industrial products.

Formaldehyde derivatives are known preservatives. For example, U.S. Pat. No. 3,987,184 discloses 1,3-dimethylol-5,5-dimethylhydantoin (DMDMH) useful as a formaldehyde donor compound for the preservation of personal care products, cosmetics, and household and industrial products. U.S. Pat. No. 5,405,862 teaches a formaldehyde donor composition containing dimethyloldimethylhydantoin, monomethyloldimethylhydantoin, and dimethylhydantoin having less than 0.1% by weight of free formaldehyde based upon 100% of total composition, useful in biocidal effective amounts in industrial or personal care products. U.S. Pat. No. 6,143,204 discloses a broad spectrum preservative composition having a dialkanol-substituted dimethyl hydantoin, an iodopropynyl compound, a stabilizer of hydantoin, and a hydroxyl solvent. U.S. Pat. No. 6,121,302 teaches a broad spectrum preservative having a dialkanol-substituted dimethyl hydantoin, one or more isothazolones, a hydantoin stabilizer and a hydroxyl solvent. Dehydroacetic acid or its salts thereof is also a known preservative, exhibiting both fungicidal and bactericidal activity.

While useful for controlling bacteria, fungi and other contamination in personal care and household products, these substances present a variety of limitations for such use including being unduly expensive; exhibiting limited anti-microbial or antifungal activity, or limited solubility in water; exhibiting undue pH dependence; adverse toxicological properties and skin sensitization or possible carcinogenicity; or they may be inactivated by commonly used materials. Furthermore, to obtain full microbiological control, a great amount of preservative must be added to the product, making it more difficult to formulate. Also, when large amounts of additive are used, the likelihood of a negative impact on that product, such as instability, odor, and breakdown of product is greater.

In light of the foregoing, it would be advantageous to formulate a broad spectrum antimicrobial composition which completely controls microbiological and fungal contamination in personal care products, household products, and industrial products. The antimicrobial composition should contain low free formaldehyde and iodine levels; and should be cost effective, stable, and suitable for use in personal care and household products. The antimicrobial composition should also be easy to formulate and effective at concentrations low enough so as not to adversely impact the product to which it is added.

### SUMMARY OF THE INVENTION

In accordance with the invention, it has now been discovered that a stable, low free formaldehyde, synergistic antimicrobial combination of a first component including one or more aldehyde donor, a second component including a stabilizer; and a third component including dehydroacetic acid or salt thereof, gives both broad spectrum bactericidal and fungicidal activity suitable for use in personal care products, household products, and various industrial products and systems. Preferably, the first component includes one or more alkanol-substituted dimethylhydantoin, and the second component includes a stabilizer of dimethyl hydantoin. Particular advantages of the antimicrobial composition of the invention are the low amounts of free formaldehyde, i.e., less than 0.2%.

By combining these components in products which require protection against microbial attack, an antimicrobial composition which completely controls microbiological contamination is obtained. Furthermore, due to the synergistic effect of the components, much less active material of each component is required as opposed to when each component is used alone. Another advantage of the synergistic antimicrobial composition is that it is able to fully control a broader spectrum of bacteria and fungi than any of the individual components. A further advantage is that the antimicrobial composition requires no iodine which at high levels is considered toxic. Accordingly, this antimicrobial composition is economical, requiring lesser amounts of expensive components, easy to use, and less likely to have toxic or skin sensitizing effects on individuals exposed to the product.

A particularly advantageous aspect of the invention is that a small amount of dehydroxyacetic acid (DHA) in combination with one or more aldehyde donor, and a stabilizer such as dimethyl hydantoin forms a stable composition. This activity could in no way be predicted based on the known reaction of dehydroacetic acid sodium salt with formaldehyde released from aldehyde donors in aqueous solution. The interaction and declining levels of dehydroacetic acid sodium salt was shown by NMR analysis in literature in the Journal of Society of Cosmetic Chemists, entitled “Dehydroacetic acid sodium salt stability in cosmetic preservative mixtures,” by C. A. Bennassi, et al., Vol. 10, pp. 29-37 (1988). This reaction forms an unstable composition, decreasing levels of DHA, thereby eliminating the fungicidal and bactericidal effect of DHA. Inclusion of a stabilizer such as dimethyl hydantoin serves to minimize the amount of free formaldehyde, thus eliminating the reaction of free formaldehyde with DHA.

Another advantage is that the antimicrobial composition of the invention remains stable and does not freeze at temperatures as low as −15° C. The more solids there are in a mixture, the more likely that the freezing point of the mixture will be increased. A typical hydantoin, and a typical hydantoin in combination with a dehydroxyacetic acid or salt thereof, freezes at −15° C. Therefore, it would be likely that the antimicrobial composition, containing more solids than a typical hydantoin or typical hydantoin in combination with DHA, would freeze at −15° C. However, the antimicrobial composition does not freeze or form crystals at −15° C.; thus, this activity could in no way be predicted as a typical hydantoin, and a typical hydantoin in combination with a dehydroacetic acid or salt thereof forms crystals at −15° C.

The antimicrobial composition may be used in personal care products such as shampoos, conditioners, rinses, creams, lotions, dental care products such as mouthwash, toothpaste, spray, and denture cleaners or soaks, baby wipes and other woven and non-woven wipes; household products such as detergents, hard surface cleaners, fabric softeners, and the like; and industrial products such as paint, wood, wood treatment, paper board, sheet rock, paper pulp, ceiling tiles, textiles, adhesives, sealants, leather, rope, plastics, petroleum, fuel, oil, and rubber and metal working fluids; or industrial systems such as pulp and papermaking processing, water treatment systems, cooling water, swimming pools and spas, decorative fountains, membranes, brewery pastures, toilet and urinal applications, food and beverage sanitation, sporicidal formulations, sterilization of clinical products and surgical instruments, and preservation including clay slurry and starch. The antimicrobial composition can be added to the aforementioned products or systems already formulated or the three components can be added to the products or systems separately.

The components of the synergistic antimicrobial composition are easy to formulate and may be added to an article or system to be treated as separate entities, or as a combination. The components are physically and chemically compatible and may be combined with carriers and excipients.

Methods for inhibiting the growth of or reducing microorganisms in personal care, household, or industrial products or systems are also provided by this invention.

Personal care products, household products, and industrial products comprising the antimicrobial composition are further provided by this invention.

### DETAILED DESCRIPTION OF THE INVENTION

As used herein, the phrases “antimicrobial,” “biocidal,” and “inhibiting microbial growth” describe the killing of, as well as the inhibition of, or control of, the growth of bacteria, yeasts, fungi, and algae.

As used herein, the phrase “microbiological contamination” describes contamination against microbes including bacteria, yeasts, fungi, and algae.

An “antimicrobial effective amount” is an amount effective to inhibit the growth of and/or kill microorganisms.

The first component of the antimicrobial composition includes one or more aldehyde donor.

### Aldehyde and Aldehyde Donor

The term “aldehyde” refers to any compound that has an aldehyde group, especially those aldehydes that exhibit antimicrobial activity, such as formaldehyde, orthophthalaldehyde, cinnamaldehyde, and mixtures thereof.

The term “aldehyde donor” as used herein is defined as any material which is not an aldehyde but upon aqueous dilution liberates a compound which gives positive reactions with aldehyde identifying reagents, i.e., a compound which can identify aldehyde groups. Generally the liberated compound has the formula:

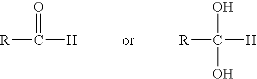

where R is any functional group. In other words, the term “aldehyde donor” includes any compound which is not an aldehyde but when hydrolyzed forms an aldehyde or a compound which gives positive reactions with aldehyde identifying reagents. Examples of aldehyde identifying reagents include, but are not limited to, Benedicts solution, Tollens reagent, and acetyl acetone.

Suitable aldehyde donors include, but are not limited to, imidazolidinyl urea, Quaternium-15, diazolindinyl urea, bromonitropropane diol, methenamine, 5-bromo-5-nitro-1,3-dioxane, sodium hydroxymethylglycinate, formalin, glutaraldehyde, polymethoxy bicyclic oxazolidine, 3,5-dimethyl-1,3,5,2H-tetrahydrothiadiazine-2-thione, hexahydro-1,3,5-tris(2-hydroxyethyl)triazine, hexahydo-1,3,5-triethyl-s-triazine, methylolhydantoins, tetrakis (hydroxymethyl) phosphonium sulfate, and any combination of any of the foregoing.

Preferred aldehyde donors include, but are not limited to alkanol-substituted dimethyl hydantoins having the formula:

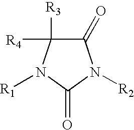

wherein R₁ and R₂ are each independently hydrogen or (CH₂)OH, with the proviso that both R₁ and R₂ cannot both be hydrogen, and R₃ and R₄ are each independently hydrogen, a methyl group, and ethyl group, a propyl group, or an aryl group.

Alkanol-substituted dimethylhydantoin compounds include those described in U.S. Pat. Nos. 3,987,184 and 4,172,140. These are condensation products of 5,5-dimethylhydantoin (DMH) with one or more moles of formaldehyde (e.g. 1,3-dimethylol-5,5-dimethylhydantoin (DMDMH), 1-methylol-5,5-dimethylhydantoin (MMDMH), or 3-methylol-5,5-dimethylhydantoin and 1-methylol-3-methyloloxymethylene-5,5-dimethylhydantoin, and mixtures thereof). Mixtures of alkanol-substituted DMH compounds can also be used. Preferred mixtures include, but are not limited to, Glydant 2000®, a 70% solution of hydantoin species including about 36% dimethylol dimethyl hydantoin (DMDMH), about 29% monomethylol dimethylhydantoin (MMDMH), and about 5% dimethyl hydantoin (DMH); and 30% water, available from Lonza Inc. of Fair Lawn, N.J. Glydant 2000® has a total formaldehyde content of 17%. Dantogard®1000 is a 70% solution of equilibrated dimethylol dimethyl hydantoin (DMDMH), monomethylol dimethylhydantoin (MMDMH), and dimethyl hydantoin (DMH), (17% total formaldehyde content), available from Lonza Inc. of Fair Lawn, N.J.

The second component includes stabilizers including hydantoins and their derivatives. The hydantoins are represented by formula:

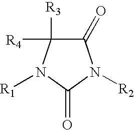

where R₁ to R₄ are independently selected from H, and a C₁ to C₆ alkyl group. Preferably the stabilizer is 5,5-dimethylhydantoin.

The stabilizers used in the invention may also include urea and its derivatives.

The third component includes dehydroacetic acid or its salts thereof; for example, dehydroacetic acid sodium salt.

Water is the preferred solvent for use in the present invention. Optionally, a hydroxyl solvent can be used which includes mono-, di-, and polyhydroxyl alcohols. For example, monohydroxyl alcohols having from about 1 to 5 carbon atoms, most preferably ethanol and propanol, may be used. Dihydroxyl alcohols (e.g., glycols) such as C₂ to C₈ diols (e.g., propylene glycol and butylene glycol) are advantageous. Other compounds which can be used include dipropylene glycol, glycerin, diglycerin, PPG-9, PPGO2-buteth-2, butoxypropanol, butoxydiglycol, PPG-2 butyl ether, glycereth-7, sorbitol, isopentyldiol, myristyl myristate, and phenoxy ethanol.

This formulation has a free formaldehyde concentration of less than 1% by weight, preferably less than 0.5, 0.2, or 0. 1% by weight. Total formaldehyde concentration is from 5% to 25% by weight and preferably from 12% to 18% by weight. Preferably, the blend contains little or no free formaldehyde. Low free formaldehyde compositions reduce workplace exposure risk to formaldehyde resulting in greater safety and reduced regulatory issues.

Table 1 provides ranges for the broad spectrum synergistic antimicrobial composition concentrates of the invention.

Broad Spectrum Synergistic Antimicrobial Composition Concentrates

| | Broad wt. % range | Preferred wt. % range |
|---|---|---|
| aldehyde donor | 5-95 | 50-75 |
| stabilizer | 0-30 | 5-20 |
| DHA | 0.5-95 | 2-30 |

The ratio of the aldehyde donor to DHA or salt thereof for the broad spectrum concentrate may broadly be from about 1:100 to 100:1, preferably from about 1:60 to 60:1, and more preferably from 0.05:30 to 30:0.05 and the ratio of stabilizer, such as dimethyl hydantoin, to aldehyde donor sufficient to minimize the amount of free formaldehyde, thus reducing or preventing the reaction of free formaldehyde with DHA.

The preservative concentrates of the invention can be readily prepared in accordance with procedures well known to those skilled in the art, simply by mixing the components set forth in Table 1, supra, and adjusting the pH using any organic or mineral acid (e.g., hydrochloric acid and acetic acid) suitable for the user's purpose. The manner in which the components are mixed can be modified to suit the needs of the formulator, as discussed below, without departing from the spirit of the invention.

The concentration of the active compounds in the use dilution depends on the nature of the microorganisms to be combated and the composition of the final product to be preserved. For example, the optimum amount of antimicrobial composition to use for preserving an aqueous composition can be determined by means of screening tests known in the art, and in accordance with the formulation ranges provided in Table 1. When preserving an aqueous composition, the use level is generally 0.00005% (0.5 ppm) to 5% (50,000 ppm) by weight, preferably from about 0.01% (100 ppm) to 1 % (10,000 ppm) of the final composition. Preservative formulations of the invention can also be used directly as they are manufactured without dilution, or in any other manner traditionally used in manufacturing, such as by metering.

Antimicrobial compositions of this invention may be used directly as they are manufactured, without dilution. They may be poured into small batches (from one to thousands of pounds) of product at any point in its manufacture. Also, the antimicrobial compositions may be pumped into medium sized batches (from thousands to tens of thousands of pounds).

The antimicrobial composition of the invention may also be metered continuously from a storage tank into large sized production runs (from tens of thousands to millions of pounds) in systems custom-designed to continuously mix all the components of the finished product at approximately the same rate that it is filled into its final package. The blending elements of continuous mixers are mostly shaped in the form of spirals or screws, effecting on rotation both a mixing and a transport of the product composition.

Because start-up is very labor-intensive, to insure all the metering equipment is properly calibrated, these systems are generally used only for very high volume, long and continuous production runs.

In another embodiment of the invention, the stabilizer may be combined with the DHA. A hydroxyl solvent may be added if desired. The resulting composition can be used in making the antimicrobial composition of the invention by mixing it with one or more aldehyde donor, such as alkanol-substituted dimethyl hydantoin.

In the antimicrobial composition containing both active ingredients, the stabilizer DMH also serves to minimize the amount of free formaldehyde in the composition. In some instances, the amount of DMH typically present in alkanol-substituted dimethyl hydantoin compositions is not sufficient to stabilize DHA where two active ingredients are used. Therefore, when formulating a stabilized antimicrobial composition of the invention, the total alkyl hydantoin concentration must be considered in determining how much alkyl hydantoin should be added to stabilize the DHA. The total alkyl hydantoin concentration is equal to free alkyl hydantoin plus reacted alkyl hydantoin (e.g., the DMH in the condensation products MMDMH and DMDMH).

The “total” aklyl hydantoin concentration is different from the “added” alkyl hydantoin concentration. Since alkyl hydantoin (free and reacted) may be present in certain alkanol-substituted dimethyl hydantoin compositions, an amount of alkyl hydantoin may be added to achieve a stabilizing amount for DHA. Thus in one embodiment alkyl hydantoin is added to a prepared alkanol-substituted dimethyl hydantoin composition (e.g., Glydant 2000®) that contains free and reacted alkyl hydantoin, such that the added alkyl hydantoin in combination with the alkylhydantoin in the alkanol-substituted dimethyl hydantoin composition provide a total alkyl hydantoin concentration that stabilizes DHA.

Glydant® is a 55% solution of 1,3-dimethylol-5,5-dimethyl hydantoin (DMDMH) available from Lonza, Inc. of Fair Lawn, N.J.

A synergistic effect is a response to a combination of two or more components that produce an effect greater than the sum of their individual effects. One method for determining whether a composition exhibits a synergistic effect is the method described in C. E. Kull et al., “Mixtures of Quaternary Ammonium Compounds and Long-chain Fatty Acids as Antifungal Agents”, Applied Microbiology, 9:538-541 (1961). The synergism value is determined by the formula:

Q_{A}/Qₐ+Q_{B}/Q_{b}

where Q_{A} is the quantity of Compound A in mixture, producing an endpoint; Qₐ is the quantity of Compound a acting alone, producing an endpoint; Q_{B} is the concentration of Compound B in the mixture, producing an endpoint. Q_{b} is the concentration of Compound b acting alone, producing an endpoint.

When the value of (Q_{A}/Qₐ+Q_{B}/Q_{b}) is less than one, the mixture is synergistic. Values for (Q_{A}/Qₐ+Q_{B}/Q_{b}) of 1 and greater than 1, represent an additive effect and an antagonistic effect, respectively. According to this method of measuring synergism, the quantity of each component in the various mixtures is compared with the quantity of pure component that is required to reach the same endpoint or to produce the same microbiological effect as the mixture.

The synergistic antimicrobial composition of the present invention is useful as an antimicrobial agent in personal care products, dental products, household products, and industrial products and systems.

The following examples are illustrative of the antimicrobial composition, however, it is understood that the invention is not limited to the specific details set forth in the examples.

TABLE 2
Freezer Stability of NaDHA.H2O in Glydant 2000 ® and Glydant ®

| | 10% NaDHA.H2O in | 10% NaDHA.H2O in |
|---|---|---|
| | Glydant 2000 ® | Glydant ® |
| 18 hours | OK-no crystals | Top ⅓: loose crystals |
| | | (frozen) |
| 48 hours | OK-no crystals | Top ⅓: loose crystals |
| | | (frozen) |

### Preservative Challenge Test Results Against Mixed Bacteria in Anionic Protein Shampoo

Mixed bacteria challenge tests were performed by adding approximately 1-5×10⁶ organisms per gram of formulation, the organisms comprising an equally divided mixture of Staphylococcus aureus (ATCC No. 6538), Pseudomonas aeruginosa (ATCC No. 9027), and Escherichia coli (ATCC No. 8739) incubated at ˜36° C. on nutrient agar slants 24 hours prior to testing. The test samples were incubated at ˜23° C. (room temperature) for the number of days indicated, after which an aliquot of the sample was taken and diluted stepwise to a 10⁶ fold reduction in concentration. The diluted samples were plated out on tryptic soy agar and incubated for 48 hours at ˜36° C. After incubation, readings of the total number of colony forming units per gram (cfu/g) were made on the samples. Table 3 illustrates the levels at which the individual components Glydant®2000 and DHA are effective and ineffective against bacteria.

TABLE 3
Preservative Challenge Test Results
against Mixed Bacteria¹ in Anionic Protein Shampoo

| % Preservative as | | | |
|---|---|---|---|
| supplied | Day 0 cfu/g. | Day 7 cfu/g. | Day 14 cfu/g. |
| 0.05% Glydant 2000 ® | 3.0 × 10⁶ | 2.4 × 10² | <10 |
| 0.025% Glydant | 3.0 × 10⁶ | 1.1 × 10⁵ | 1.3 × 10⁷ |
| 2000 ® | | | |
| 0.0125% Glydant | 3.0 × 10⁶ | 3.0 × 10⁷ | 7.3 × 10⁷ |
| 2000 ® | | | |
| 0.20% DHA | 3.0 × 10⁶ | 1.0 × 10³ | <10 |
| 0.150% DHA | 3.0 × 10⁶ | 8.0 × 10³ | 1.0 × 10⁶ |
| 0.025% Glydant | 3.0 × 10⁶ | 2.0 × 10³ | <10 |
| 2000 ® + 0.075% | | | |
| DHA | | | |
| Unpreserved | 3.0 × 10⁶ | 3.0 × 10⁷ | 3.0 × 10⁷ |

cfg/g = Colony forming units in 1 gram.
¹An equal mixture of Staphylococcus aureus (ATCC No. 6538), Pseudomonas aeruginosa (ATCC No. 9027), and Escherichia coli (ATCC No. 8739).

TABLE 4
Synergy at Day 14

| Sample | Q_{A} | Q_{B} | Qₐ | Q_{b} | $\frac{{Q}_{A}}{{Q}_{a}} + \frac{{Q}_{B}}{{Q}_{b}}$ | $\frac{< 1 =}{Synergy}$ |
|---|---|---|---|---|---|---|
| 0.05% | | | 0.05% | | | |
| Glydant | | | Glydant 2000 ® | | | |
| 2000 ® | | | | | | |
| 0.2% DHA | | | | 0.2% | | |
| | | | | DHA | | |
| 0.025% Glydant 2000 ® +0.075% DHA | 0.025% | 0.075% | | | $\frac{0.025}{0.05} + \frac{0.075}{0.2}$ | 0.5 + 0.37 =0.87 Synergy |

Thus, the antimicrobial composition described herein provides a method for inhibiting the growth of or reducing microorganisms such as bacteria and fungi in a wide variety of compositions, i.e., personal care products, household products, and industrial products and systems. The antimicrobial composition eliminates the need for iodine, while at the same time utilizing ultra-low free formaldehyde compositions in combination with DHA or DHA.Na which is fungicidal and bactericidal.

All patents, applications, articles, publications, and test methods mentioned above are hereby incorporated by reference.

Many variations of the present invention will suggest themselves to those skilled in the art in light of the above detailed description. Such obvious variations are within the full intended scope of the appended claims.

We claim:

1. An antimicrobial composition comprising a synergistic antimicrobial effective amount of a mixture of:
(a) a first component including one or more aldehyde donor;
(b) a second component including a stabilizer; and
(c) a third component including a dehydroacetic acid or salt thereof.

2. The composition of claim 1 wherein the dehydroacetic acid salt is dehydroacetic acid sodium salt.

3. The composition of claim 1 wherein the aldehyde donor is an alkanol-substituted dimethyl hydantoin selected from the group consisting of 1,3-dimethylol-5,5-dimethylhydantoin, 1-methylol-5,5-dimethylhydantoin, 3-methylol-5,5-dimethylhydantoin, 1-methylol-3-methyloloxymethylene-5,5-dimethylhydantoin, 1,3-dimethyloloxymethylene-5,5-dimethylhydantoin, or mixtures thereof.

4. The composition of claim 3, wherein the alkanol-substituted dimethyl hydantoin is a mixture of dimethyloldimethylhydantoin and monomethyloldimethylhydantoin.

5. The composition of claim 1, wherein the aldehyde donor is selected from the group consisting of imidazolidinyl urea, Quaternium-15, diazolindinyl urea, bromonitropropane diol, methenamine, 5-bromo-5-nitro-1,3-dioxane, sodium hydroxymethylglycinate, formalin, glutaraldehyde, polymethoxy bicyclic oxazolidine, 3,5-dimethyl-1,3,5,2H-tetrahydrothiadiazine-2-thione, hexahydro-1,3,5-tris(2-hydroxyethyl)triazine, hexahydo-1,3,5-triethyl-s-triazine, methylolhydantoins, tetrakis (hydroxymethyl) phosphonium sulfate, or mixtures thereof.

6. The composition of claim 1 wherein the stabilizer is dimethyl hydantoin or derivative thereof.

7. The composition of claim 1, wherein the mixture is substantially free of iodine.

8. The composition of claim 1, wherein the mixture has a free formaldehyde concentration of less than 0.2% by weight, based on 100% weight of the mixture.

9. The composition of claim 1 wherein the first component is present in the mixture in an amount of between about 20% to about 95% by weight, based on 100% by weight of the mixture.

10. The composition of claim 1 wherein the second component is present in the mixture an amount of between about 0% to about 30% by weight, based on 100% weight of the mixture.

11. The composition of claim 1 wherein the third component is present in the mixture in an amount of between about 0.5% to about 40% by weight, based on 100% weight of the mixture.

12. A method of preparing a synergistic antimicrobial composition which comprises blending 20 to 95 parts of an aldehyde donor and 5 to 20 parts of a stabilizer or derivative thereof to form a homogeneous mixture, mixing a solvent and 1 to 40 parts of DHA or salt thereof with the foregoing mixture to obtain a homogeneous solution containing a total formaldehyde content of at least 2% and less than 0.2% free formaldehyde.

13. A method of inhibiting the growth of or reducing microorganisms comprising applying a synergistically antimicrobial effective amount of the composition of claim 1.

14. A method of inhibiting the growth of or reducing microorganisms in personal care products such as shampoos, conditioners, rinses, creams, lotions, cosmetics, soaps, dental products such as mouthwash, toothpaste, spray, denture cleaners and denture soaks, baby wipes and other woven and non-woven wipes; household products such as laundry detergents, hard surface cleaners, fabric softeners; and industrial products such as paint, wood, wood treatment, paper board, sheet rock, paper pulp, ceiling tiles, textiles, adhesives, sealants, leather, rope, plastics, petroleum, fuel, oil, and rubber and metal working fluids; comprising applying an effective amount of the composition of claim 1 to the personal care product, household product, or industrial product.

15. A method of inhibiting the growth of or reducing microorganisms in industrial systems such as pulp and papermaking processing; water treatment systems; cooling water; swimming pools and spas; decorative fountains; membranes; brewery pastures; toilet and urinal applications; food and beverage sanitation; sporicidal formulations; sterilization of clinical products and surgical instruments, and preservation, including clay slurry and starch, comprising applying an effective amount of the composition of claim 1 to the industrial system.

16. A personal care, household, or industrial product comprising the composition of claim 1.

17. A personal care, household, or industrial product which comprises an effective amount of a mixture of a first component including one or more aldehyde donor, and a second component including dimethyl hydantoin, and a third component including dehydroacetic acid.

18. The product of claim 17 wherein the product is a household or industrial product selected from the group consisting of fabric softeners, laundry detergents, hard surface cleaners, paint, wood, wood treatment, paper board, sheet rock, paper pulp, ceiling tiles, textiles, adhesives, sealants, leather, rope, plastics, petroleum, fuel, oil, and rubber and metal working fluids.

19. The product of claim 17 wherein the product is a personal care product selected from the group consisting of shampoos, conditioners, rinses, creams, lotions, cosmetics, soaps, mouthwash, toothpaste, spray, denture cleaners and denture soaks, baby wipes and other woven and non-woven wipes.
